(19) United States
(12) Patent Application Publication
(10) Pub. No.: US 2007/0162118 A1
(43) Pub. Date: Jul. 12, 2007

(54) Posterior chamber phakic intraocular lens

(76) Inventors: George W. Rozakis, Lakewood, OH (US); Igor G. Valyunin, Milwaukee, WI (US)

Correspondence Address: FRED ZOLLINGER III, P.O. BOX 2368, NORTH CANTON, OH, 44720, US

(21) Appl. No.: 11/637,475

(22) Filed: Dec. 11, 2006

Related U.S. Application Data
(63) Continuation of application No. 10/988,157, filed on Nov. 12, 2004.
(60) Provisional application No. 60/580,424, filed on Jun. 17, 2004.
(60) Provisional application No. 60/519,978, filed on Nov. 14, 2003.

(51) Int. Cl. A61F 2/16

(52) U.S. Cl. 623/6.44; 623/6.49

(57) ABSTRACT

A phakic intraocular lens includes a body having a flat anterior-facing surface and a curved posterior-facing surface that defines the optical power of the lens. The lens allows fluid to flow between the phakic lens and the iris and between the phakic lens and the natural lens. An enlarged outer rim helps maintain the position of the lens. Some embodiments are provided with openings, channels, or both at the circumferential edge of the flat anterior-facing surface or the lens body to help prevent the flat surface of the lens from sealing against the iris.

### CROSS REFERENCE TO RELATED APPLICATIONS

This application is a continuation of U.S. patent application Ser. No. 10/988,157 filed 12 Nov. 2004, which claims the benefit of U.S. Provisional Patent Application Ser. No. 60/580,424 filed Jun. 17, 2004, and U.S. Provisional Patent Application Ser. No. 60/519,978 filed Nov. 14, 2003; the disclosures of which are incorporated herein by reference.

### BACKGROUND OF THE INVENTION

1. Technical Field

This invention generally relates to an intraocular lens and, more particularly, to a posterior chamber, phakic intraocular lens. Specifically, the present invention is directed to a phakic intraocular lens having a flat front surface and a curved rear surface at the optical portion of the lens.

2. Background Information

Various posterior chamber, phakic intraocular lenses are known in the art. These lenses are implanted directly behind the iris in front of the eye's natural lens. One drawback with these lenses is the need for an iridotomy that allows fluid to flow from the posterior chamber to the anterior chamber of the eye. The art desires an implant that may be used without an iridotomy. Another drawback with known lenses is the limitation on the size of the optical portion of the lens. The art desires a lens with a large optical portion. The art also desires a lens having a configuration that does not interfere with the fluid flow patterns in the eye while having a structure that maintains a desired location within the eye. Typical known lenses use haptics that span the eye chamber and engage opposed portions of the ciliary bodies to wedge the lens in place. Other lenses use the iris to create centering forces on the lens. The art desires a phakic lens that does not relay on as much contact with the eye to remain in a desired position as known lenses.

### SUMMARY OF THE INVENTION

The invention provides a phakic intraocular lens having a flat front surface and a curved rear optical surface to define the optical power of the lens. The lens may be used with or without an iridotomy. The lens has positioning arms that help maintain the position of the lens within the eye. Different configurations for the positioning arms are disclosed. In one embodiment, the positioning arms are short and cannot wedge into opposed portions of the ciliary bodies. In one embodiment of the invention, the rear surface of the positioning arms has a radius of curvature substantially equal to the radius of curvature of the front surface of the natural lens of the eye. The invention also provides a lens having an optical body and a pair of positioning arms wherein the configuration of the lens provides additional space for aqueous behind the lens to help keep the lens spaced from the natural lens.

The invention also provides a phakic intraocular lens having a flat front surface and a curved rear optical surface to define the optical power of the lens. The lens has an enlarged rim disposed about the optical portion that maintains the lens within a desired position within the eye.

The invention also provides a lens having a joint between the optical portion and positioning arms with the joint defining channels that prevent the iris from forming a seal with the lens when the lens engages the iris. In another embodiment of the invention, openings are provided in the optical portion and/or the positioning arms.

Another aspect of the invention is the method of designing the lens based on the measurements of the eye.

### BRIEF DESCRIPTION OF THE DRAWINGS

FIG. 1 is a sectional view of the eye having a phakic intraocular lens implanted next to the natural lens.

FIG. 2 is a front elevation view of a first embodiment of the lens of the invention.

FIG. 3 is a section view taken along line 3-3 of FIG. 2.

FIG. 4 is a front elevation view of the first embodiment of the lens with opposed channels defined between the flat front surface and the positioning arms.

FIG. 5 is a section view taken along line 5-5 of FIG. 4.

FIG. 5A is an enlarged view of the encircled portion of FIG. 5.

FIG. 6 is a section view, taken from the side, of the first embodiment of the phakic lens and the natural lens showing the relative radii of the rear surface of the phakic lens, the rear surface of the positioning arms, and the front surface of the natural lens.

FIG. 7 is a section view, taken from the side, of a second embodiment of the phakic lens and the natural lens showing the relative radii of the rear surface of the phakic lens, the rear surface of the positioning arms, and the front surface of the natural lens.

FIG. 8 is a section view, taken from the side, of a third embodiment of the phakic lens and the natural lens showing the relative radii of the rear surface of the phakic lens, the rear surface of the positioning arms, and the front surface of the natural lens.

FIG. 9 is a section view, taken from the side, of a fourth embodiment of the phakic lens and the natural lens showing the relative radii of the rear surface of the phakic lens, the rear surface of the positioning arms, and the front surface of the natural lens.

FIG. 10 shows a comparison of a prior art lens to the lens of the invention with the same optical power.

Similar numbers refer to similar elements throughout the specification.

### DESCRIPTION OF THE PREFERRED EMBODIMENTS

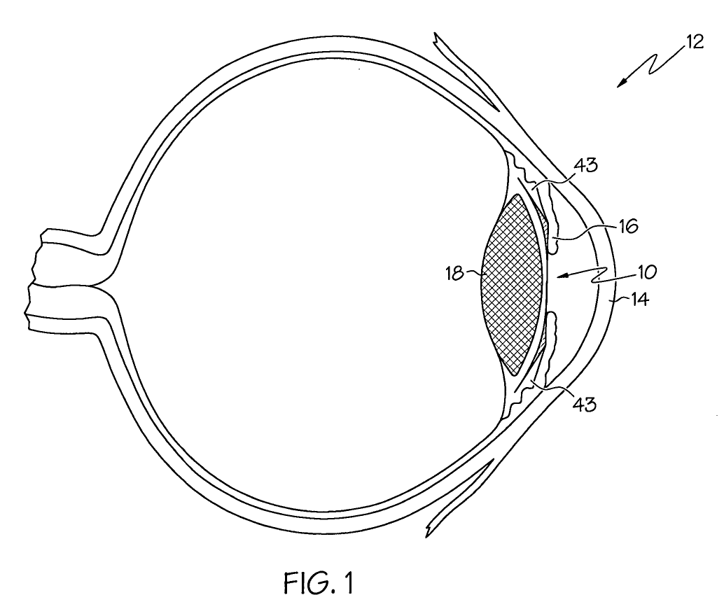

A phakic intraocular lens made in accordance with the concepts of the present invention is indicated generally by the numeral 10 in the drawing figures. Lens 10 is positioned in the posterior chamber of the eye in FIG. 1. Eye 12 includes a cornea 14, an iris 16 and a natural lens 18. Phakic intraocular lens 10 is positioned behind iris 16 and in front of lens 18 so that it influences the light entering natural lens 18 of eye 12.

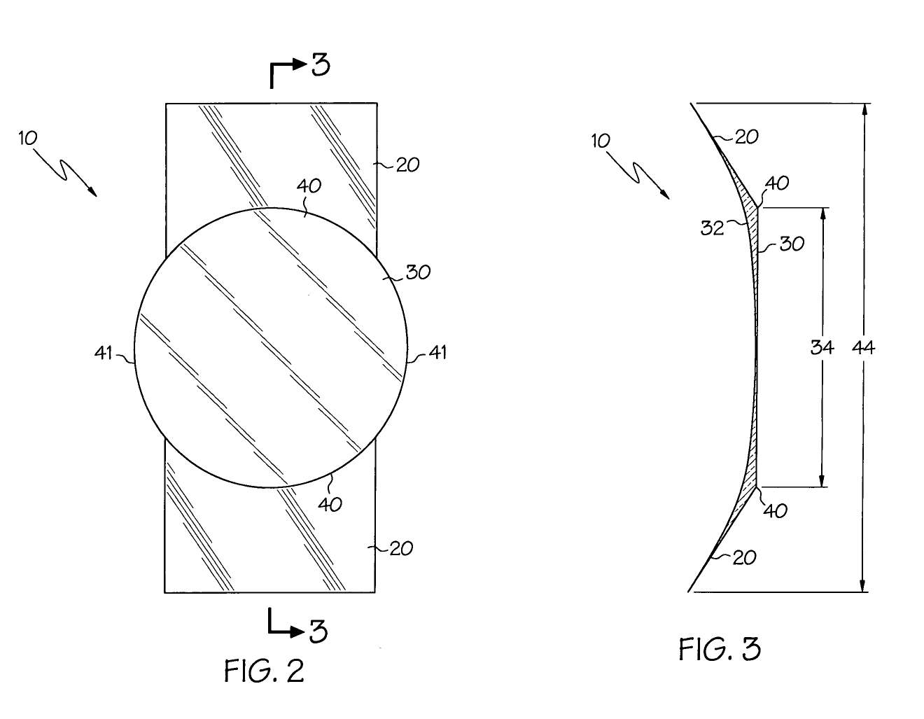

At least a pair of positioning arms 20 extends from opposite sides of lens 10 to help maintain the position of lens 10 with respect to eye 12. The edges of positioning arms 20 may be rounded to provide a round surface for contact with the zonules. Arms 20 are not designed to be wedged into opposed portions of the ciliary body but may contact the ciliary body or the zonules in different lens sizes and configurations. Numerous types and shapes for positioning arms 20 are known in the art and any of the variety will function with the lens of the present invention. Positioning arms 20 may be rectangular when viewed in elevation as shown in FIG. 2. In other embodiments of the invention, three or four positioning arms 20 are equally-spaced about the optical portion of lens 10. In another embodiment, positioning arms 20 extend entirely about the optical portion of the lens.

In each of the lens embodiments described in this application, the lens body has a flat front optical surface 30 and a curved rear optical surface 32. Front surface 30 provides a large flat surface disposed directly behind iris 16 so that lens 10 will smoothly slide against the rear of iris 16 in the event that lens 10 is moved into contact with iris 16. In most embodiments, the diameter 34 of flat front surface 30 is 6 mm to 9 mm. In some embodiments, diameter 34 may be reduced to 4 mm. The radius of curvature 36 of curved rear optical surface 32 is 14 mm to 21 mm. The specific dimensions may be determined by measuring the optical correction for the patient and then measuring the eye of the patient. This measurement may be performed with ultrasound. Lens 10 is designed after the physical dimensions of the natural lens and iris are known. For example, the overall diameter of the space between the natural lens and iris may be measured and dimension 44 may then be design to prevent lens 10 from wedging itself in place. The curvature of the anterior surface of lens 18 may also be measured to determine curvature 42 that properly vaults lens 18. In the context of this application the term “flat surface” includes lens structures that have an anterior surface that is slightly curved to prevent undesirable reflections through the pupil. This curvature is insignificant to the optical properties of the lens and thus falls with the definition of “flat surface” as used in this specification.

Positioning arms 20 are connected to the optical portion of lens 10 at the outer circumference or portions of the outer circumference of the optical portion. Positioning arms 20 may be provided in a wide variety of shapes when view from the front elevation as shown in FIG. 2. One rectangular embodiment is shown in FIG. 2 wherein the width of the rectangle is smaller than the diameter of the optical portion of lens 10. In another embodiment, the width of the rectangle is equal to the diameter of the optical portion. The connection between the positioning arm 20 and the optical portion of the lens is referred to as the joint 40 of lens 10 even though the optical portion and positioning arms 20 are integrally formed.

Another feature of lens 10 is that the outer diameter of the optical portion of the lens is the thickest area (in cross section) of lens 10 and forms a bulbous rim 41 about the circumference of the optical portion. Rim 41 does not, however, protrude from the flat front surface of lens 10. This area is referred to as the outer rim 41. Rim 41 corresponds to the joints 40 at the locations of the positioning arms 20. The thick rim 41 is positioned in the gap 43 defined between natural lens 18, iris 16, the ciliary body, and the zonules that support lens 18. The thick, bulbous rim 41 maintains the general position of lens 10 with surface 30 behind the pupil. Rim 41 also functions to prevent lens 10 from slipping through the zonules into the vitreous. Rim 41 also allows the eye to create centering forces on lens 10 when the eye interacts with rim 41.

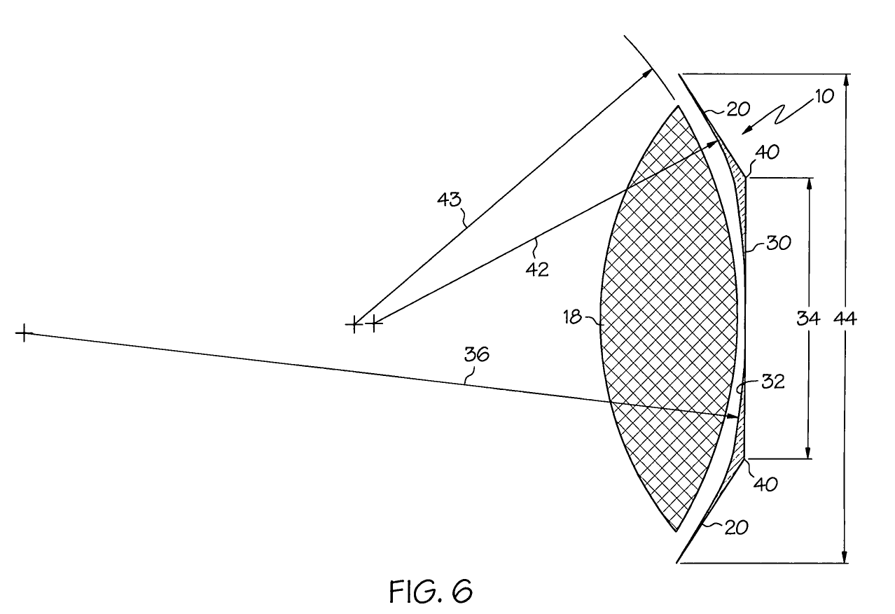

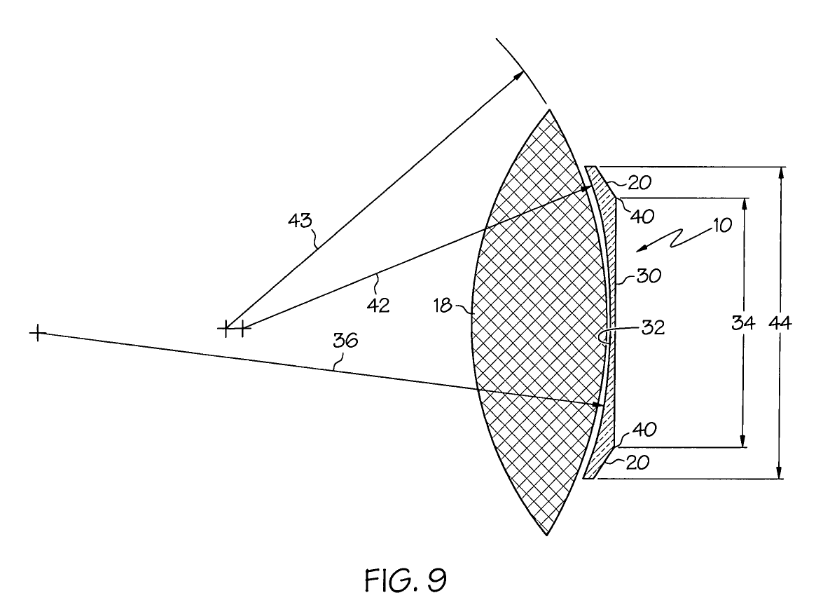

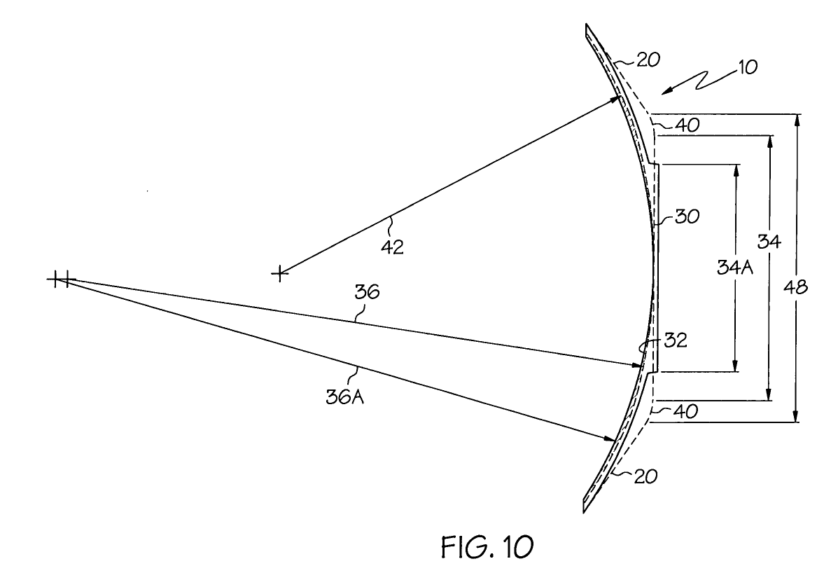

The radius of curvature 42 of the positioning arms 20 is smaller than the radius 36 of curved rear optical surface 32. This arrangement vaults the rear surface of lens 10 away from natural lens 18 thus allowing space for the aqueous to flow between lens 10 and natural lens 18. The extra space provided may be seen in the exemplary comparison of FIG. 10 wherein like-powered lens are overlaid to illustrate the extra aqueous space behind lens 10. The additional space is hatched in FIG. 10. The exemplary lens illustrated I FIG. 10 is one of the type disclosed in U.S. Pat. No. 6,015,435. Lens 10 provides significantly more room between lens 18 and the rear surface of lens 10. This room allows the aqueous of the eye to flow freely between lens 10 and lens 18 to help keep lens 10 from contacting lens 18. In one embodiment of the invention, radius 42 is equal to the radius of curvature 43 of natural lens 18. The tip-to-tip length 44 of positioning arms 20 is greater than the outer diameter of natural lens 18 in the embodiments of FIGS. 6, 7, and 8. In FIG. 9, positioning arms 20 are short and have a tip-to-tip length that is shorter than the outer diameter of lens 18. In the FIG. 9 embodiment, rim 41 may be larger and more prominent to provide the centering forces. Further, the FIG. 9 embodiment will not constantly engage the zonules disposed about lens 18.

FIGS. 2, 3, and 6 depict an embodiment of the invention wherein the positioning arms 20 are tapered from the joint to the tip with the joint defines a substantially sharp corner. FIG. 6 shows one embodiment wherein radius 36 is 20 mm, radius 42 is 10 mm, and radius 43 is 10 mm. The diameter 34 of the optical portion is 7 mm. The tip-to-tip dimension 44 is 12 mm.

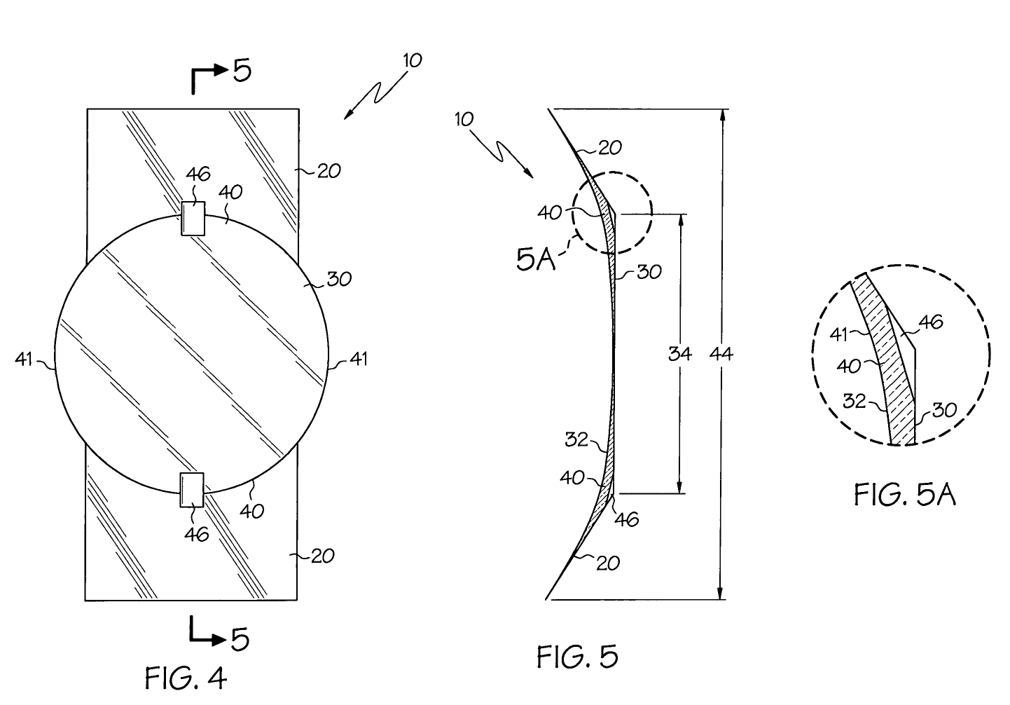

FIGS. 4 and 5 depict an embodiment similar to FIGS. 2 and 3 except that the joint of FIGS. 4 and 5 defines channels 46 that prevent joint 40 of lens 10 from forming a seal with iris 16. Channels 46 do not pass entirely through the body of lens 10 as shown in FIG. 5A. In other embodiments of the invention, through openings may be provided at joints 40 or in positioning arms 20. In another embodiment, a small opening is provided in the center of the optical portion. This central opening may have a diameter of 0.8 mm. These channels and openings allow aqueous to flow freely.

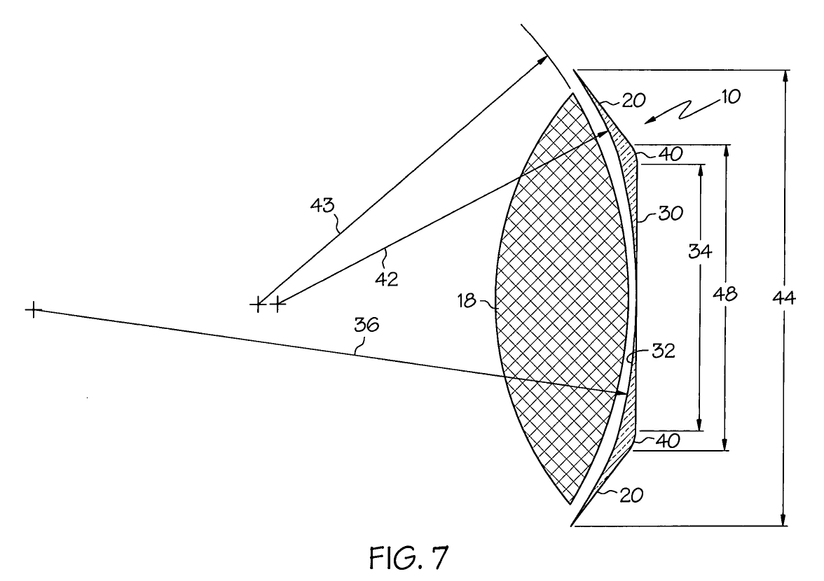

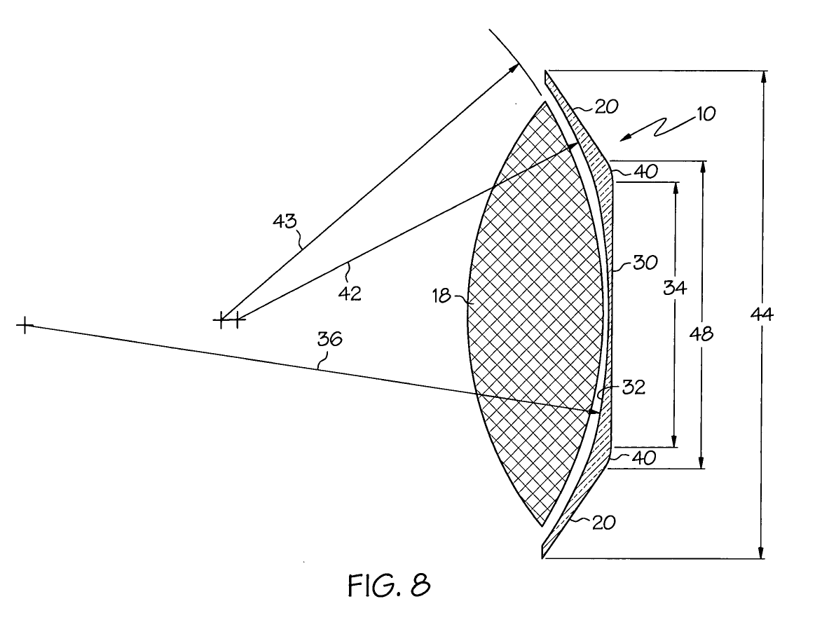

FIG. 7 depicts an alternative embodiment wherein joint 40 defines radiused corners. FIG. 7 shows one embodiment wherein radius 36 is 14.9 mm, radius 42 is 10 mm, and radius 43 is 10 mm. The diameter 34 of the optical portion (inside the radiused corners) is 7.17 mm with the diameter 48 of the optical portion (outside the radiused corners) being 8.03 mm. The tip-to-tip dimension 44 is 11.88 mm.

FIG. 8 depicts an alternative embodiment wherein the joint defines radiused corners. FIG. 8 shows one embodiment wherein radius 36 is 18 mm, radius 42 is 10 mm, and radius 43 is 10 mm. The diameter 34 of the optical portion (inside the radiused corners) is 6.98 mm with the diameter 48 of the optical portion (outside the radiused corners) being 7.18 mm. The tip-to-tip dimension 44 is 12 mm. In FIG. 8, the positioning arms have a substantially constant thickness adjacent their outer ends and flat outer ends.

FIG. 9 depicts an alternative embodiment wherein the joint defines radiused or smoothly-rounded corners. FIG. 9 shows one embodiment wherein radius 36 is 14.9 mm, radius 42 is 10 mm, and radius 43 is 10 mm. The diameter 34 of the optical portion (inside the radiused corners) is 6.02 mm. The tip-to-tip dimension 44 is 8 mm.

The lens embodiments of the invention are preferably fabricated from an acrylic. However, various lens materials are known in the art. For instance, it is know that the optical portions of intraocular lenses may be fabricated from polymethyl methacrylate, poly-2-hydroxyethyl methacrylate, methyl methacrylate copolymers, siloxanylalkyl, fluoroalkyl and aryl methacrylate, silicone, silicone elastomers, polysulfones, polyvinyl alcohols, polyethylene oxides, copolymers of fluoroacrylates and methacrylate, and polymers and copolymers of hydroxyalkyl methacrylate, such as 2-hydroxyethyl methacrylate, as well as methacrylic acid, acrylic acid, acrylamide methacrylamide, N,N-dimethylacrylamide, and N-vinylpryrrolidone. Additionally, compounds that absorb ultraviolet or other short wavelength (e.g. below about 400 nm) radiation, such compounds derived from benzotriazole groups, benzophenone groups, or mixtures thereof may be added to the monomers and/or polymers that constitute the implant. Other compounds well known in the art may also be used in fabricated optical portion of lens 10 of the present invention.

The advantages of the invention are that the flat front surface of the lens can have a larger diameter than lenses with curved front surfaces. The large diameter and large radius of the posterior optical surface allow the lens to be formed in a wide range of optical powers such as those that are needed by patients who are inilligeble for corneal laser surgery. The large diameter optical portion also minimizes halos. The large flat surface minimizes pressure on the iris so that fluid may flow from the posterior chamber to the anterior chamber of the eye. Further, the channels of the invention allow fluid flow even when the joint of the lens contacts the iris. The lens may thus be implanted without an iridotomy. The thick rim disposed about the optical portion of the lens maintains the lens in the desired location.

The lens may be implanted be folding the lens and slipping the folded lens through the pupil of the eye.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention is an example and the invention is not limited to the exact details shown or described.

1. A phakic intraocular refractive correction lens for implanting in a posterior chamber of an eye intermediate a natural crystalline lens and an iris of the eye, the phakic intraocular lens comprising:
a lens body having an optical portion and at least a pair of positioning arms;
the lens body having a shape predetermined with respect to a shape of the natural crystalline lens and iris to form a spacing between a posterior surface of the lens body and an anterior surface of the natural crystalline lens at a location between the natural crystalline lens and the pupil of the eye;
the optical body having an anterior surface and a posterior surface; the anterior surface being flat; the posterior surface having a radius of curvature; the optical body having an thin central portion and a thick outer rim;
the thick outer rim disposed intermediate the iris and the natural crystalline lens;
the thin central portion of the optical body defining an opening adapted to allow fluid to flow through the optical portion of the lens body;
the positioning arms extending outwardly and rearwardly from the thick outer rim;
the optical body being disposed intermediate the positioning arms;
each of the positioning arms having a curved posterior surface having a radius of curvature; and
the radius of curvature of each of the posterior surfaces of the positioning arms being less than the radius of curvature of the posterior surface of the optical body.

2. The lens of claim 1, wherein no portion of the lens body protrudes forwardly from the anterior surface of the optical body.

3. The lens of claim 1, wherein the thick outer rim has an anterior surface that defines a channel.

4. The lens of claim 1, wherein the thick outer rim defines an opening.

5. The lens of claim 1, wherein the flat anterior surface of the optical body has a diameter of between 4 mm and 9 mm.

6. The lens of claim 5, wherein the flat anterior surface of the optical body has a diameter of between 6 mm and 9 mm.

7. The lens of claim 1, wherein each of the positioning arms has an anterior surface; the outer rim having an anterior surface; and the anterior surfaces of positioning arms being disposed tangent to the anterior surface of the outer rim.

8. A phakic intraocular refractive correction lens for implanting in a posterior chamber of an eye intermediate a natural crystalline lens and an iris of the eye, the phakic intraocular lens comprising:
a lens body having an optical portion and at least a pair of positioning arms;
the lens body having a shape predetermined with respect to a shape of the natural crystalline lens and iris to form a spacing between a posterior surface of the lens body and an anterior surface of the natural crystalline lens at a location between the natural crystalline lens and the pupil of the eye;
the optical body having an anterior surface and a posterior surface; the anterior surface being flat; the posterior surface having a radius of curvature; the optical body having an thin central portion and a thick outer rim;
the flat anterior surface of the optical body having a diameter of 6 to 9 mm;
the radius of curvature of the posterior surface of the optical body being in the range of 14 to 21 mm;
the thin central portion of the optical body defining an opening adapted to allow fluid to flow through the optical portion of the lens body;
the positioning arms extending outwardly and rearwardly from the thick outer rim;
no portion of the lens body protruding forwardly from the anterior surface of the optical body;
the optical body being disposed intermediate the positioning arms;
each of the positioning arms having a curved posterior surface having a radius of curvature; and
the radius of curvature of each of the posterior surfaces of the positioning arms being less than the radius of curvature of the posterior surface of the optical body.

9. The lens of claim 8, wherein each of the positioning arms has an anterior surface; the outer rim having an anterior surface; and the anterior surfaces of positioning arms being disposed tangent to the anterior surface of the outer rim.

10. A phakic intraocular refractive correction lens for implanting in a posterior chamber of an eye intermediate a natural crystalline lens and an iris of the eye, the phakic intraocular lens comprising:
a lens body having an optical portion and at least a pair of positioning arms;
the lens body having a shape predetermined with respect to a shape of the natural crystalline lens and iris to form a spacing between a posterior surface of the lens body and an anterior surface of the natural crystalline lens at a location between the natural crystalline lens and the pupil of the eye;
the optical body having an anterior surface and a posterior surface; the anterior surface being flat; the posterior surface having a radius of curvature; the optical body having an thin central portion and a thick outer rim;
the optical body defining an opening that allows fluid to flow through the optical body of the lens;
lens body having a shape configured to float within the posterior chamber of the eye with the thick outer rim disposed intermediate the posterior surface of the pupil and the anterior surface of the natural lens; and
the lens body being located entirely in the posterior chamber of the eye floating in the aqueous humor between the iris and the natural lens, and wherein said lens body does not include a structure for permanent fixation in the eye.

11. The lens of claim 10, wherein no portion of the lens body protrudes forwardly from the anterior surface of the optical body.

12. The lens of claim 10, wherein the thick outer rim has an anterior surface that defines a channel.

13. The lens of claim 10, wherein the thick outer rim defines an opening.

14. The lens of claim 10, wherein the flat anterior surface of the optical body has a diameter of between 4 mm and 9 mm.

15. The lens of claim 14, wherein the flat anterior surface of the optical body has a diameter of between 6 mm and 9 mm.

16. The lens of claim 10, wherein each of the positioning arms has an anterior surface; the outer rim having an anterior surface; and the anterior surfaces of positioning arms being disposed tangent to the anterior surface of the outer rim.
